(19) United States
(12) Patent Application Publication
(10) Pub. No.: US 2024/0315580 A1
(43) Pub. Date: Sep. 26, 2024

(54) PULSE WAVE ESTIMATION DEVICE AND PULSE WAVE ESTIMATION METHOD

(71) Applicant: Mitsubishi Electric Corporation, Tokyo (JP)

(72) Inventors: Ryohei MURACHI, Tokyo (JP); Yudai NAKAMURA, Tokyo (JP)

(73) Assignee: Mitsubishi Electric Corporation, Tokyo (JP)

(21) Appl. No.: 18/272,614

(22) PCT Filed: Feb. 19, 2021
(86) PCT No.: PCT/JP2021/006237

§ 371 Date: Jul. 17, 2023

(51) Int. Cl. A61B 5/024

(52) U.S. Cl. CPC A61B 5/024

(57) ABSTRACT

A pulse wave estimation device includes: to acquire first and second image frames and of a subject; to detect a facial organ point indicating a position of a facial organ of the subject in the first image frame; to set a tracking point indicating a position of the facial organ in the second image frame by referring to the first image frame; to set a position of a measurement region in the first image frame on the basis of the facial organ point in the first image frame; to set a position of the measurement region in the second image frame on the basis of the tracking point in the second image frame; to measure a luminance value of a pixel in the measurement region; and to estimate a pulse wave of the subject on the basis of a luminance difference that is a difference between the luminance values.

### TECHNICAL FIELD

The present disclosure relates to a pulse wave estimation device and a pulse wave estimation method.

### BACKGROUND ART

In the biological information measurement device described in Patent Literature 1 that measures condition of a living body, for example, a blood pressure in a non-contact manner, it is necessary to set a skin area for measuring the blood pressure in an image of a facial organ in each of a plurality of image frames in which the living body is imaged. In order to set the skin area, the biological information measurement device performs, for example, facial organ detection processing of detecting a position of the facial organ on a first image frame. On the other hand, the biological information measurement device performs tracking processing on a plurality of image frames subsequent to the first image frame, instead of performing the facial organ detection processing. The tracking processing is processing of setting the position of the skin area in the target image frame by referring to the position of the skin area in the previous image frame.

When movement occurs in the facial organ of the living body between the image frames before and after the tracking processing, for example, when change in the facial expression, movement of the position of the face, or the like occurs, a deviation occurs between the position of the skin area in the image frame before the tracking processing, that is, before the movement of the facial organ occurs, and the position of the skin area in the image frame after the tracking processing, that is, after the movement of the facial organ occurs. Therefore, when the tracking processing is sequentially performed on a plurality of consecutive image frames, the above-described deviation is accumulated. In order to eliminate the accumulated deviation, that is, to reset it, the biological information measurement device performs reset processing, which is the facial organ detection processing, at intervals of a predetermined number of image frames.

### CITATION LIST

### Patent Literature

Patent Literature 1: JP 2016-190022 A

### SUMMARY OF INVENTION

### Technical Problem

However, the biological information measurement device has a problem. The problem is that the accumulated deviation occurs between the position of the skin area detected by the facial organ detection processing in the first image frame and the position of the skin area detected by the tracking processing in another image frame on which the reset processing is not performed because the another image frame follows the first image frame without separating from the first image frame by not less than the predetermined number of image frames described above, due to the tracking processing being sequentially performed between the first image frame and the another image frame.

The biological information measurement device has another problem. The problem is that, in the image frames before and after the reset processing, the accumulated deviation occurs between the position of the skin area having the accumulated deviation in the image frame before the reset processing and the position of the skin area having no deviation as a result of the reset processing in the image frame after the reset processing.

An object of the present disclosure is to provide a pulse wave estimation device that solves at least one of the two problems described above.

### Solution to Problem

In order to solve the above problem, a pulse wave estimation device according to the present disclosure includes: an acquisition unit to acquire a plurality of consecutive image frames of a subject whose pulse wave is to be estimated, the image frames including a first image frame which is before a second image frame and the second image frame which is after the first image frame; a detection unit to detect a facial organ point indicating a position of a facial organ of the subject in the first image frame; a tracking unit to set a tracking point indicating a position of the facial organ in the second image frame by referring to the first image frame; a first setting unit to set a position of a measurement region in the first image frame on a basis of the detected facial organ point in the first image frame, the measurement region being a region where a luminance value for estimating the pulse wave is to be measured; a second setting unit to set a position of the measurement region in the second image frame on a basis of the set tracking point in the second image frame; a measurement unit to measure a luminance value of a pixel in the set measurement region in the first image frame and a luminance value of a pixel in the set measurement region in the second image frame; and an estimation unit to estimate the pulse wave of the subject on a basis of a luminance difference that is a difference between the measured luminance value of the measurement region in the first image frame and the measured luminance value of the measurement region in the second image frame.

### Advantageous Effects of Invention

According to the pulse wave estimation device of the present disclosure, it is possible to avoid a situation in which an accumulated deviation occurs as the tracking processing is sequentially performed. According to the pulse wave estimation device of the present disclosure, it is possible to avoid a situation in which an accumulated deviation occurs between image frames before and after the reset processing. Therefore, the pulse wave estimation device according to the present disclosure can estimate the pulse wave of the subject with higher accuracy than a pulse wave estimation device in which at least one of the two accumulated deviations occurs.

### BRIEF DESCRIPTION OF DRAWINGS

FIG. 1 is a functional block diagram of a pulse wave estimation device 1 according to an embodiment.

FIG. 2 illustrates operations of a photographing unit 11 and a detection unit 12 according to the embodiment.

FIG. 3 illustrates a configuration of a setting unit 13 according to the embodiment.

FIG. 4 illustrates an operation of a facial organ detecting unit 13A according to the embodiment.

FIG. 5 illustrates an operation of a tracking unit 13B according to the embodiment.

FIG. 6 illustrates a configuration of the pulse wave estimation device 1 according to the embodiment.

FIG. 7 is a flowchart illustrating an operation of the pulse wave estimation device 1 according to the embodiment.

FIG. 8 is a time chart illustrating the operation of the pulse wave estimation device 1 according to the embodiment.

FIG. 9 illustrates an operation of a pulse wave estimation device of a comparative example.

### DESCRIPTION OF EMBODIMENTS

An embodiment of a pulse wave estimation device according to the present disclosure will be described.

### First Embodiment

### Embodiment

### <Configuration of Embodiment>

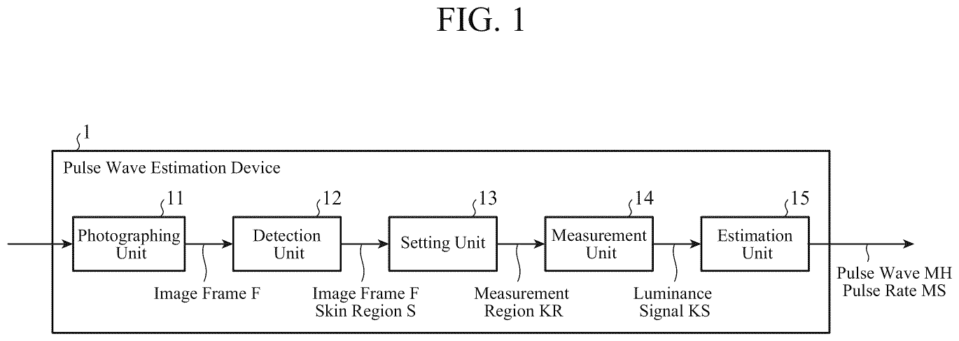

FIG. 1 is a functional block diagram of a pulse wave estimation device 1 according to an embodiment.

Hereinafter, the functions of the pulse wave estimation device 1 of the embodiment will be described with reference to FIG. 1.

As illustrated in FIG. 1, the pulse wave estimation device 1 according to the embodiment includes a photographing unit 11, a detection unit 12, a setting unit 13, a measurement unit 14, and an estimation unit 15 in order to estimate a pulse wave on the basis of luminance of a pixel in a photographed image.

The photographing unit 11 corresponds to an “acquisition unit”, the measurement unit 14 corresponds to a “measurement unit”, and the estimation unit 15 corresponds to an “estimation unit”. The correspondence relationship of the setting unit 13 will be described later.

As described later, the detection unit 12 that detects a skin region does not correspond to a “detection unit” that detects a facial organ point. On the other hand, a facial organ detecting unit 13A in the setting unit 12 that performs facial organ detection processing and tracking processing corresponds to the “detection unit” and also corresponds to a “first setting unit”.

Hereinafter, in order to facilitate description and understanding, reference numerals in which suffixes attached to the reference numerals are omitted may be used as a generic term. For example, image frames F1 to Fm may be collectively referred to as an image frame F, and skin regions S1 to Sm may be collectively referred to as a skin region S.

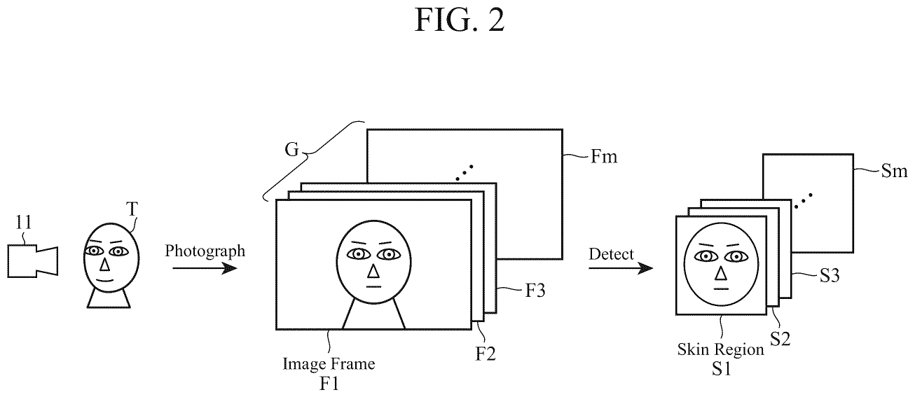

FIG. 2 illustrates operations of the photographing unit 11 and the detection unit 12 according to the embodiment.

The photographing unit 11 is, for example, a camera, and photographs an image G of a subject T whose pulse wave is to be estimated, for example, the image G centering on the upper body of the subject T, in particular, the face of the subject T, as illustrated in FIG. 2. As illustrated in FIG. 2, the image G includes a plurality of consecutive image frames F1, F2, F3, . . . , and Fm. Here, m is an integer equal to or greater than 2.

For example, the image frame F1 is before the image frame F2, and the image frame F2 is after the image frame F1. Similarly, the image frame F2 is before the image frame F3, and the image frame F3 is after the image frame F2.

The photographing unit 11 outputs the plurality of photographed image frames F1 to Fm to the detection unit 12.

As illustrated in FIG. 2, the detection unit 12 detects the skin region S1 from the image frame F1, for example. The skin region S1 is a rectangular region representing the position, shape, size, and the like of the entire face of the subject T in the image frame F1.

The detection unit 12 detects the skin regions S2, S3, . . . , Sm from the other image frames F2, F3, . . . , Fm similarly to the detection of the skin region S1 from the image frame F1.

Hereinafter, in order to facilitate description and understanding, for example, both the skin region S1 itself and the information indicating the skin region S1 are collectively referred to as a skin region S1. The same applies to the other skin regions S2 to Sm.

The skin regions S1 to Sm may represent other parts of the subject T, for example, positions of the neck, shoulders, arms, hands, and the like of the subject T, instead of the face of the subject T described above. The skin regions S1 to Sm may represent the position and the like of a part of the face of the subject T, for example, a part including one or more of the forehead, eyebrows, eyes, nose, mouth, cheeks, chin, and the like of the face of the subject T instead of the entire face of the subject T. In the skin regions S1 to Sm, the number of the entire face of the subject T and the part of the face of the subject T is not limited. More specifically, the skin regions S1 to Sm may represent only one, for example, the position of the entire face of the subject T, or the like, or may represent two, for example, the position of the right cheek of the subject T and the position of the left cheek of the subject T, or the likes, or may further represent three, for example, the position of the nose of the subject T, the position of the mouth of the subject T, and the position of the chin of the subject T, or the likes.

Returning to FIG. 1, the detection unit 12 outputs the plurality of image frames F1 to Fm and the plurality of skin regions S1 to Sm to the setting unit 13.

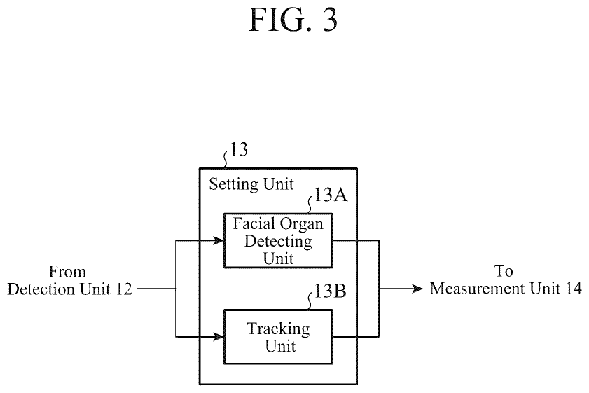

FIG. 3 illustrates a configuration of the setting unit 13 according to the embodiment.

As illustrated in FIG. 3, the setting unit 13 includes the facial organ detecting unit 13A and a tracking unit 13B. The facial organ detecting unit 13A corresponds to the “detection unit” and the “first setting unit”, and the tracking unit 13B corresponds to a “tracking unit” and a “second setting unit”.

In order to make it possible to calculate a luminance difference between two preceding and following frames, for example, a luminance difference between the image frame F2 and the immediately following image frame F3, the face detecting unit 13A performs the facial organ detection processing on the preceding image frame F2.

Similarly to the face detecting unit 13A, the tracking unit 13B makes it possible to calculate a luminance difference between two preceding and following frames; however, the tracking unit 13B is different from the facial organ detecting unit 13A. For example, in order to make it possible to calculate a luminance difference between the image frame F2 and the immediately preceding image frame F1, the tracking unit 13B performs the tracking processing on the following image frame F2.

To sum up the facial organ detection processing by the facial organ detecting unit 13A and the tracking processing by the tracking unit 13B, the facial organ detection processing and the tracking processing are performed for each of the image frames F2, F3, . . . .

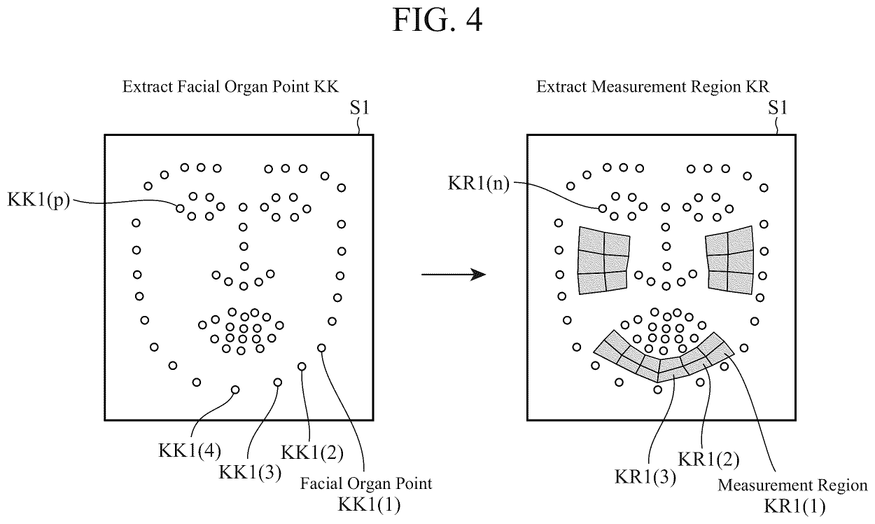

For example, the facial organ detecting unit 13A sets, in the skin region S1 received from the detection unit 12, one or more rectangular measurement regions KR1(1) to KR1(n) (illustrated in FIG. 4) in which luminance for estimating a pulse wave of the subject T is to be measured. Here, n is an integer equal to or greater than 1.

FIG. 4 illustrates an operation of the facial organ detecting unit 13A according to the embodiment.

The facial organ detecting unit 13A uses, for example, a constrained local model (CLM), which is a model of a conventionally known facial organ detecting method. The facial organ detecting unit 13A performs the facial organ detection processing using the CLM. Specifically, the facial organ detecting unit 13A detects the coordinate values of a plurality of facial organ points KK1(1) to KK1(p) in the skin region S1, for example, as illustrated in FIG. 4 (in the left frame). Here, the facial organ points KK1(1) to KK1(p) are feature points for determining the face of the subject T. p is an integer equal to or greater than 2.

Subsequent to the facial organ detection processing, as illustrated in FIG. 4 (in the right frame), the facial organ detecting unit 13A sets the coordinate values of the measurement regions KR1(1) to KR1(n) using the facial organ points KK1(1) to KK1(p) detected for the skin region S1 as references (FIG. 4 (in the left frame)).

Also for the skin regions S2 to Sm other than the skin region S1, the facial organ detecting unit 13A performs processing similar to the processing performed for the skin region S1, that is, detects the coordinate values of the facial organ point KK and sets the coordinate values of the measurement region KR.

For example, the facial organ detecting unit 13A detects coordinate values of a plurality of facial organ points KK2(1), KK2(2), . . . (Not shown) in the skin region S2, and sets the coordinate values of a plurality of measurement regions KR2(1), KR2(2), . . . (Not shown).

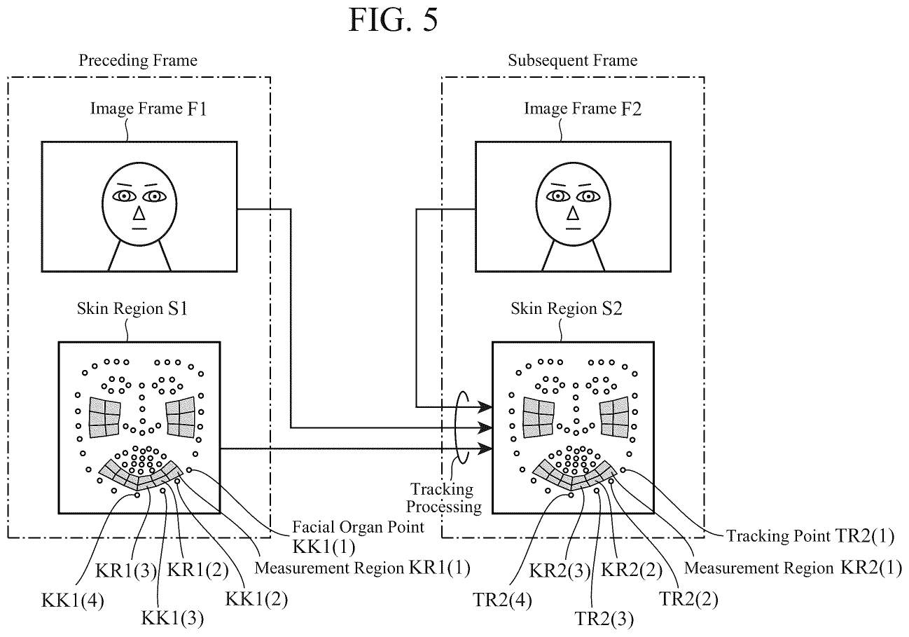

FIG. 5 illustrates an operation of the tracking unit 13B of the embodiment.

The tracking unit 13B performs the tracking processing between preceding and following image frames using a Kanade-Lucas-Tomasi (KLT) tracker, which is a conventionally known tracking technology.

As illustrated in FIG. 5, the tracking unit 13B performs the tracking processing on the basis of, for example, the image frame F1, the skin region S1 in the image frame F1, and the image frame F2 subsequent to the image frame F1. As a result, as illustrated in FIG. 5, the tracking unit 13B sets coordinate values of tracking points TR2(1), TR2(2), TR2(3), TR2(4), . . . , which are feature points for determining the face of the subject T, in the skin region S2 of the image frame F2.

Subsequent to the tracking processing, as illustrated in FIG. 5, the facial organ detecting unit 13B sets the coordinate values of measurement regions KR2(1), KR2(2), KR2(3), . . . using the tracking points TR2(1), TR2(2), TR2(3), TR2(4), . . . as references.

Comparing the facial organ detecting unit 13A with the tracking unit 13B, the facial organ detecting unit 13A uses the above-described facial organ points KK1(1) to KK1(p) (illustrated in FIG. 4) as references, while the tracking unit 13B uses the above-described tracking points TR2(1), TR2(2), TR2(3), TR2(4), . . . (illustrated in FIG. 5) as references.

Also for the skin regions S3 to Sm other than the skin region S2, the tracking unit 13B performs processing similar to the processing performed for the skin region S2, that is, sets the coordinate values of the tracking point TR and sets the coordinate values of the measurement region KR.

As illustrated in FIG. 1, the setting unit 13 outputs the measurement regions KR, that is, the measurement regions KR1(1), KR1(2), . . . (illustrated in FIG. 4), KR2(1), KR2(2), . . . (illustrated in FIG. 5), KRm(1), KRm(2), . . . (Not shown) for the skin regions S1 to Sm to the measurement unit 14.

Returning to FIG. 1, the description of the configuration of the pulse wave estimation device 1 will be continued.

The measurement unit 14 measures the luminance value of the pixel in each of the measurement regions KR of the preceding and following image frames F received from the setting unit 13, and generates a luminance signal indicating a difference between the measured luminance values in time series.

For example, for the image frames F1 and F2 illustrated in FIG. 5, the measurement unit 14 calculates, for example, a difference between an average value of the luminance values of a plurality of pixels included in the measurement region KR1(1) in the image frame F1 and an average value of the luminance values of a plurality of pixels included in the measurement region KR2(1) which is corresponding to the measurement region KR1(1) of the image frame F1 and which is in the image frame F2.

Subsequently, the measurement unit 14 calculates a difference between an average value of the luminance values of a plurality of pixels included in the measurement region KR2(1) in the image frame F2 and an average value of the luminance values of a plurality of pixels included in the measurement region KR3(1) (Not shown) which is corresponding to the measurement region KR2(1) of the image frame F2 and which is in the image frame F3 (Not shown).

Subsequently, the measurement unit 14 further calculates a difference between an average value of the luminance values of a plurality of pixels included in the measurement region KR3(1) in the image frame F3 and an average value of the luminance values of a plurality of pixels included in the measurement region KR4(1) (Not shown) which is corresponding to the measurement region KR3(1) of the image frame F3 and which is in the image frame F4 (Not shown).

The measurement unit 14 may use, for example, a variance value instead of the average value described above.

The measurement unit 14 performs the same processing as described above also on the measurement regions KR of the image frames F other than the measurement regions KR1(1), KR2(1), KR3(1), and KR4(1) of the image frames F1, F2, F3, and F4 described above. As illustrated in FIG. 1, the measurement unit 14 outputs a luminance signal KS indicating a pixel difference between the measurement regions KR of the preceding and subsequent image frames F to the estimation unit 15.

The estimation unit 15 estimates a pulse wave MH on the basis of the luminance signal KS received from the measurement unit 14. The estimation unit 15 further calculates a pulse rate MS on the basis of the estimated pulse wave MH. The estimation unit 15 outputs at least one of the estimated pulse wave MH and the calculated pulse rate MS.

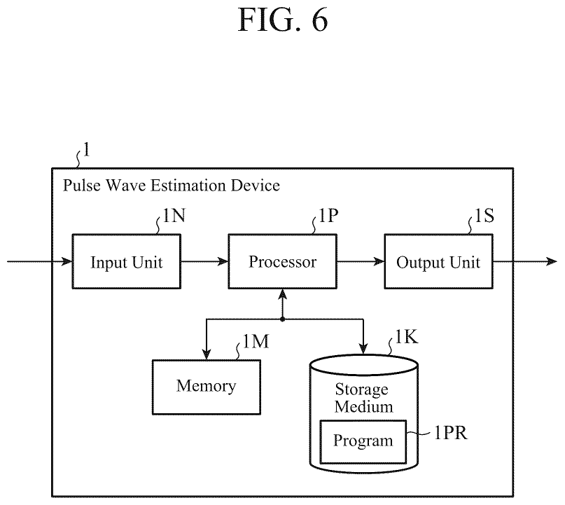

FIG. 6 illustrates a configuration of the pulse wave estimation device 1 according to the embodiment.

As illustrated in FIG. 6, the pulse wave estimation device 1 according to the embodiment includes an input unit IN, a processor 1P, an output unit 1S, a storage medium 1K, and a memory 1M to perform the above-described functions.

The input unit IN includes, for example, a camera, a microphone, a keyboard, a mouse, or a touch panel. The processor 1P is a core of a well-known computer that operates hardware in accordance with software. The output unit 1S includes, for example, a liquid crystal monitor, a printer, or a touch panel. The memory 1M includes, for example, a dynamic random access memory (DRAM) or a static random access memory (SRAM). The storage medium 1K includes, for example, a hard disk drive (HDD), a solid state drive (SSD), or a read only memory (ROM).

The storage medium 1K stores a program 1PR. The program 1PR is a command group that defines contents of processing to be executed by the processor 1P.

Regarding the relationship between the functions and the configuration of the pulse wave estimation device 1, the processor 1P executes, on hardware, the program 1PR stored in the storage medium 1K using the memory 1M, and controls the operations of the input unit IN and the output unit 1S as necessary, thereby implementing the functions of the respective units including the photographing unit 11 to the estimation unit 15.

### <Operation of Embodiment>

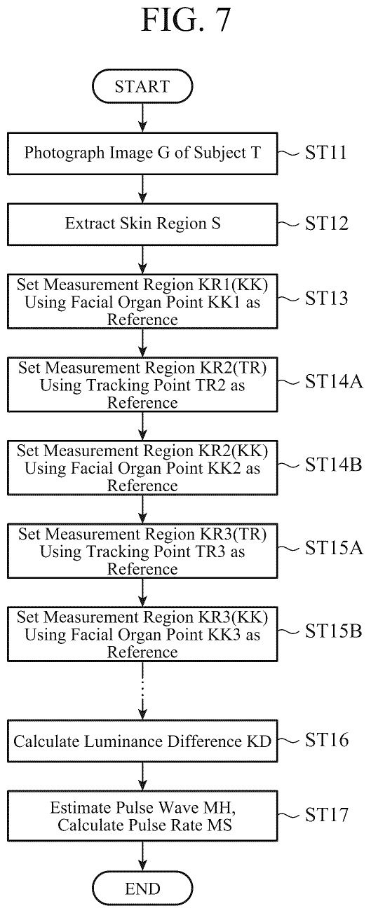

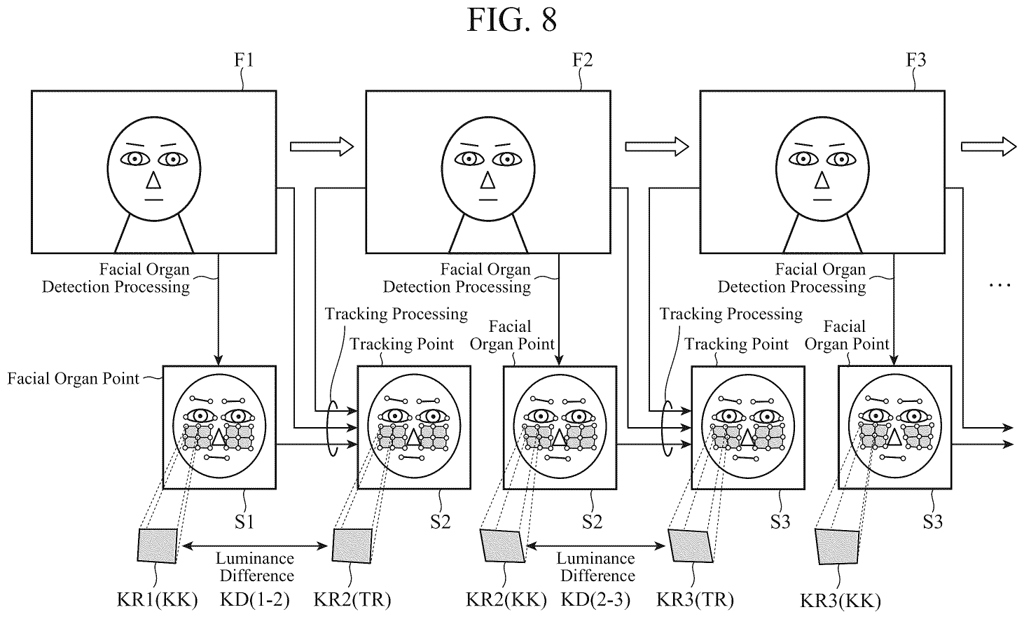

FIG. 7 is a flowchart illustrating the operation of the pulse wave estimation device 1 of the embodiment. FIG. 8 is a time chart illustrating the operation of the pulse wave estimation device 1 according to the embodiment. Hereinafter, the operation of the pulse wave estimation device 1 will be described with reference to the flowchart of FIG. 7 and the time chart of FIG. 8.

Hereinafter, in order to facilitate description and understanding, suffixes “(KK)” and “(TR)” are attached to the measurement regions KR. The suffix “(KK)” means the facial organ detection processing, and on the other hand, the suffix “(TR)” means the tracking processing.
Step ST11: As illustrated in FIG. 2, the photographing unit 11 photographs the image G of the subject T whose pulse wave is to be estimated, that is, a plurality of image frames F1 to Fm. The photographing unit 11 outputs the plurality of photographed image frames F1 to Fm to the detection unit 12.
Step ST12: As illustrated in FIG. 2, the detection unit 12 extracts the plurality of skin regions S1 to Sm from the plurality of image frames F1 to Fm. The detection unit 12 sequentially outputs the plurality of image frames F1 to Fm and the plurality of skin regions S1 to Sm to the setting unit 13.
Step ST13: When the image frame F1 is output from the detection unit 12, in the setting unit 13 the facial organ detecting unit 13A detects coordinate values of a plurality of facial organ points KK1(Not shown) in the skin region S1 of the image frame F1 as illustrated in FIG. 8 since the image frame F1 is the first image frame. The facial organ detecting unit 13A also sets the coordinate values of the measurement region KR1(KK) in the skin region S1 using the detected coordinate values of the plurality of facial organ points KK1 as references, for example, as illustrated in FIG. 8.
Step ST14A: When the image frame F2 subsequent to the image frame F1 is output from the detection unit 12, in the setting unit 13 the tracking unit 13B first performs the tracking processing on the basis of the image frame F1, the skin region S1, and the image frame F2 as illustrated in FIG. 8 since the image frame F2 is the second image frame. As a result, the tracking unit 13B sets a plurality of tracking points TR2(Not shown) in the skin region S2 in the image frame F2. As illustrated in FIG. 8, the tracking unit 13B sets the coordinate values of the measurement region KR2(TR) to correspond to the measurement region KR1(KK) using the plurality of set tracking points TR2 as references.
Step ST14B: Next, in the setting unit 13, as illustrated in FIG. 8, the facial organ detecting unit 13A detects coordinate values of a plurality of facial organ points KK2(Not shown) in the skin region S2 of the image frame F2. The facial organ detecting unit 13A also sets the coordinate values of the measurement region KR2(KK) in the skin region S2 using the detected coordinate values of the plurality of facial organ points KK2 as references, as illustrated in FIG. 8.
Step ST15A: When the image frame F3 is output from the detection unit 12, in the setting unit 13 the tracking unit 13B first performs the tracking on the basis of the image frame F2, the skin region S2, and the image frame F3 as illustrated in FIG. 8 similarly to step ST14A since the image frame F3 is the third image frame. As a result, the tracking unit 13B sets a plurality of tracking points TR3 (Not shown) in the skin region S3 in the image frame F3. The tracking unit 13B sets coordinate values of the measurement region KR3(TR) to correspond to the measurement region KR2(KK) using the plurality of set tracking points TR3 as references.
Step ST15B: Next, in the setting unit 13, as in step ST14B, the facial organ detecting unit 13A detects coordinate values of facial organ points KK3 (Not shown) in the skin region S3 of the image frame F3 as illustrated in FIG. 8. The facial organ detecting unit 13A also sets the coordinate values of the measurement region KR3(KK) in the skin region S3 using the detected coordinate values of the facial organ points as references.

The subsequent image frames F4, F6, . . . , and Fm are also processed in the same manner as in steps ST15A and ST15B described above.

Step ST16: The measurement unit 14 measures the luminance value of the measurement region KR, and specifically measures the measurement region KR1(KK), the measurement region KR2(TR), the measurement region KR2(KK), the measurement region KR3(TR), . . . .

The measurement unit 14 calculates a luminance difference KD after the measurement described above. For example, the measurement unit 14 calculates an average value of the luminance values of the pixels in the measurement region KR1(KK) illustrated in FIG. 8 and an average value of the luminance values of the pixels in the measurement region KR2(TR) illustrated in FIG. 8, and further calculates a difference between both the average values, that is, a luminance difference KD(1-2). Similarly, for example, the measurement unit 14 calculates an average value of the luminance values of the pixels in the measurement region KR2(KK) illustrated in FIG. 8 and an average value of the luminance values of the pixels in the measurement region KR3(TR) illustrated in FIG. 8, and further calculates a difference between both the average values, that is, a luminance difference KD(2-3). The measurement unit 14 similarly calculates the luminance difference KD between the other image frames F3, F4, . . . . The measurement unit 14 outputs the luminance signal KS indicating the luminance difference KD(1-2), the luminance difference KD(2-3), . . . .

Step ST17: When the luminance signal KS is output from the measurement unit 14, the estimation unit 15 estimates the pulse wave MH on the basis of the luminance signal KS. The estimation unit 15 also calculates the pulse rate MS on the basis of the estimated pulse wave MH. The estimation unit 15 outputs at least one of the estimated pulse wave MH and the calculated pulse wave number MS.

### Effects of Embodiment

As described above, according to the pulse wave estimation device 1 of the embodiment, the facial organ detecting unit 13A sets the measurement region KR1(KK) on the basis of the facial organ points KK1 for the image frame F1. Furthermore, the tracking unit 13B sets the measurement region KR2(TR) on the basis of the tracking points TR2 for the image frame F2 subsequent to the image frame F1, and the facial organ detecting unit 13A sets the measurement region KR2(KK) on the basis of the facial organ points KK2 for the image frame F2. Furthermore, the measurement regions KR(KK) and KR(TR) are similarly set for subsequent image frames F3 to Fm. After the setting described above, the measurement unit 14 measures the pixel values in the measurement regions KR(KK) and KR(TR), and calculates the luminance difference KD of the measured pixel values between the preceding and subsequent measurement regions KR. Then, the estimation unit 15 estimates the pulse wave MH and calculates the pulse wave number MS on the basis of the luminance differences KD(1-2), KD(2-3), . . . .

As a result, it is possible to avoid a situation in which the deviation continues to be accumulated as the tracking continues to be sequentially performed. In addition, it is also possible to avoid a situation in which the accumulated deviation occurs between image frames before and after the conventional reset processing.

As a result, the luminance difference KD between the measurement regions KR1(KK) and KR2(TR), the luminance difference KD between KR2(KK) and KR3(TR), . . . can be measured with higher accuracy than before. As a result, the estimation of the pulse wave MH of the subject T and the calculation of the pulse wave number MS can be performed with higher accuracy than before.

### Comparative Example

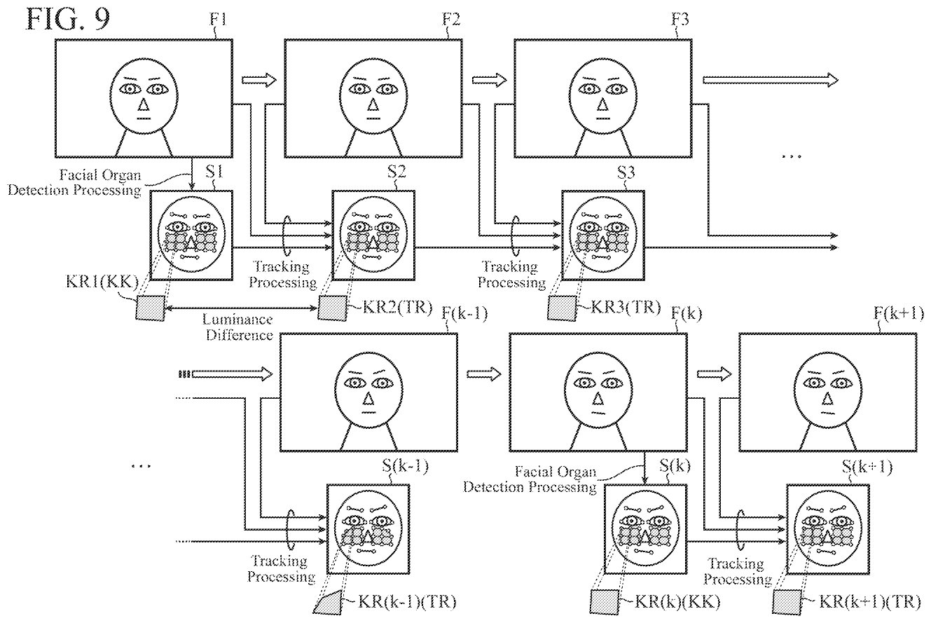

FIG. 9 illustrates an operation of a pulse wave estimation device of a comparative example.

In the pulse wave estimation device of the comparative example, as illustrated in FIG. 9, the measurement region KR1(KK) is set by performing the facial organ detection processing only on the first image frame F1, and on the other hand, the measurement regions KR2(TR), KR3(TR), . . . are set by performing the tracking processing without performing the facial organ detection processing on the subsequent plurality of other image frames F2, F3, . . . .

More specifically, a plurality of facial organ points KK1(Not shown) are detected by performing the facial organ detection processing on the image frame F1 as the first image frame, and the measurement region KR1(KK) is set in the skin region S1 using the plurality of detected facial organ points KK1 as references.

In the pulse wave estimation device of the comparative example, the tracking processing is performed on the image frame F2 subsequent to the image frame F1 on the basis of the image frame F1, the skin region S1, and the image frame F2 without performing the facial organ detection processing, so that a plurality of tracking points TR2(Not shown) are detected. Then, the measurement region KR2(TR) is set in the skin region S2 using the detected tracking points TR2 as references. In the pulse wave estimation device of the comparative example, the measurement regions KR3(TR), . . . , and KR(k−1) (TR) are set by continuing the tracking processing similarly to the image frame F2 also for the image frames F3, . . . , and F(k−1) subsequent to the image frame F2.

On the other hand, in the pulse wave estimation device of the comparative example, it is determined that the tracking processing should be stopped, that is, the tracking processing should be reset, at intervals of approximately k (k is a predetermined positive integer) image frames. Accordingly, in the pulse wave estimation device of the comparative example, the tracking processing is stopped, that is, the tracking processing is reset for the image frame F(k), and then the facial organ detection processing is performed for the image frame F(k) similarly to the facial organ detection processing performed for the image frame F1. As a result, a plurality of facial organ points KK (k) (Not shown) are detected, and the measurement region KR(k) (KK) is set in the skin region S (k) using the plurality of detected facial organ points KK (k) as references. In the pulse wave estimation device of the comparative example, the measurement region KR(k+1) (TR) and the like are set by continuing the tracking processing for the image frame F(k+1) and the like subsequent to the image frame F(k) similarly to the image frame F2 and the like subsequent to the image frame F1.

In the pulse wave estimation device of the comparative example, as described above, the facial organ detection processing is performed on the image frames F1, F(k), . . . , and on the other hand, the tracking processing is performed on other image frames F2, F3, . . . subsequent to the image frame F1, the image frame F(k+1) subsequent to the image frame F(k), and the like without performing the facial organ detection processing. As a result, in the pulse wave estimation device of the comparative example, the tracking processing is sequentially performed in such a manner as the tracking processing for the image frame F2, the tracking processing for the image frame F3, the tracking processing for the image frame F4 (Not shown), . . . . As the tracking processing is sequentially performed, the deviation between the measurement region KR2(TR) and the measurement region KR3(TR), the deviation between the measurement region KR3(TR) and the measurement region KR4(TR), . . . , are accumulated. As a result, for example, the position of the measurement region KR(k−1) (TR) of the image frame F(k−1) immediately before the image frame F(k) on which the facial organ detecting processing is performed is separated from the position of the measurement region KR1(KK) of the first image frame F1 by a distance corresponding to the accumulated deviation.

In the pulse wave estimation device of the comparative example, the position of the measurement region KR(k) (KK) for the image frame F(k) on which the tracking processing is reset, that is, only the facial organ detection processing is performed is substantially the same as the position of the measurement region KR1(KK) of the image frame F1 on which only the facial organ detection processing is performed. Therefore, the position of the measurement region KR(k) (KK) for the image frame F(k) and the position of the measurement region KR(k−1) (TR) for the image frame F(k−1) are separated by a distance substantially equal to the above-described distance between the position of the measurement region KR1(KK) and the position of the measurement region KR(k−1) (TR).

In contrast to the pulse wave estimation device of the comparative example, the pulse wave estimation device 1 of the embodiment basically performs the facial organ detection processing and the tracking processing for each image frame F of the image frames F1, F2, F3, . . . as described above. Therefore, the pulse wave estimation device 1 of the embodiment can avoid a situation in which the above-described distance resulting from the accumulation of the deviation occurs between the position of the measurement region KR(k−1) (TR) of the image frame F(k−1) and the position of the measurement region KR1(KK) of the first image frame F1. Similarly, it is possible to avoid a situation in which a distance substantially equal to the above-described distance also occurs between the position of the measurement regions KR(k−1) (TR) of the image frame F(k−1) and the position of the measurement region KR(k) (KK) of the image frame F(k).

### Modifications

### <Modifications>

Instead of performing the facial organ detection processing and the tracking processing described above for each image frame F, that is, for each of the image frames F1, F2, F3, . . . , the pulse wave estimation device 1 of the modification may perform the facial organ detection processing and the tracking processing at intervals of a predetermined number of image frames F only and perform only the tracking processing for the other image frames F. More specifically, for example, the pulse wave estimation device 1 of the modification may perform the facial organ detection processing on the image frame F1, and then, for the subsequent image frames F, perform only the tracking processing on the image frames F2 and F3, perform both the facial organ detection processing and the tracking processing on the image frame F4, perform only the tracking processing on the image frames F5 and F6, and perform both the facial organ detection processing and the tracking processing on the image frame F7.

Any component of the embodiment can be modified or omitted.

### INDUSTRIAL APPLICABILITY

The pulse wave estimation device and the pulse wave estimation method according to the present disclosure can be used, for example, for estimation of a pulse wave and calculation of a pulse wave number.

### REFERENCE SIGNS LIST

1: pulse wave estimation device, 11: photographing unit, 12: detection unit, 13: setting unit, 14: measurement unit, 15: estimation unit, 13A: facial organ detecting unit, 13B: tracking unit, IN: input unit, 1P: processor, 1S: output unit, 1M: memory, 1K: storage medium, 1PR: program, T: subject, G: image, F: image frame, S: skin region, KK: facial organ point, TR: tracking point, KR: measurement region, KD: luminance difference, KS: luminance signal, MH: pulse wave, MS: pulse rate.

1. A pulse wave estimation device, comprising:
a processor to execute a program; and
a memory to store the program which, when executed by the processor, performs processes of,
acquiring a plurality of consecutive image frames of a subject whose pulse wave is to be estimated, the image frames including a first image frame which is before a second image frame and the second image frame which is after the first image frame;
detecting a facial organ point indicating a position of a facial organ of the subject in the first image frame;
setting a tracking point indicating a position of the facial organ after movement of the facial organ in the second image frame by referring to the first image frame;
setting a position of a measurement region in the first image frame on a basis of the detected facial organ point in the first image frame, the measurement region being a region where a luminance value for estimating the pulse wave is to be measured;
setting a position of the measurement region in the second image frame on a basis of the set tracking point in the second image frame;
measuring a luminance value of a pixel in the set measurement region in the first image frame and a luminance value of a pixel in the set measurement region in the second image frame; and
estimating the pulse wave of the subject on a basis of a luminance difference that is a difference between the measured luminance value of the measurement region in the first image frame and the measured luminance value of the measurement region in the second image frame.

2. The pulse wave estimation device according to claim 1, wherein detecting the facial organ point and setting the tracking point are performed for each of the image frames.

3. The pulse wave estimation device according to claim 1, wherein detecting the facial organ point and setting the tracking point are performed at intervals of a predetermined number of image frames.

4. A pulse wave estimation method, comprising:
acquiring a plurality of consecutive image frames of a subject whose pulse wave is to be estimated, the image frames including a first image frame which is before a second image frame and the second image frame which is after the first image frame;
detecting a facial organ point indicating a position of a facial organ of the subject in the first image frame;
setting a tracking point indicating a position of the facial organ after movement of the facial organ in the second image frame by referring to the first image frame;
setting a position of a measurement region in the first image frame on a basis of the detected facial organ point in the first image frame, the measurement region being a region where a luminance value for estimating the pulse wave is to be measured;
setting a position of the measurement region in the second image frame on a basis of the set tracking point in the second image frame;
measuring a luminance value of a pixel in the set measurement region in the first image frame and a luminance value of a pixel in the set measurement region in the second image frame; and
estimating the pulse wave of the subject on a basis of a luminance difference that is a difference between the measured luminance value of the measurement region in the first image frame and the measured luminance value of the measurement region in the second image frame.
